(19) United States
(12) Patent Application Publication
(10) Pub. No.: US 2022/0218776 A1
(43) Pub. Date: Jul. 14, 2022

(54) COMPOSITION FOR USE IN A METHOD FOR PREVENTION OR TREATMENT OF MALTNUTRITION IN A SUBJECT SUFFERING FROM A CHRONIC INFLAMMATORY BOWEL DISEASES

(71) Applicant: SOFAR S.P.A., Trezzano Rosa (MI) (IT)

(72) Inventor: Andrea BIFFI, Trezzano Rosa [MI] (IT)

(21) Appl. No.: 17/611,880

(22) PCT Filed: Jun. 5, 2020
(86) PCT No.: PCT/IB2020/055311

§ 371 Date: Nov. 16, 2021

(30) Foreign Application Priority Data
Jun. 7, 2019 (IT) 102019000008394

(51) Int. Cl. A61K 36/45; A61K 38/17; A61K 47/44; A61K 31/702; A61K 33/00; A61K 33/06; A61K 33/26; A61K 33/34; A61K 33/32; A61K 33/18; A61K 33/24; A61K 33/04; A61K 31/07; A61K 31/593; A61K 31/355; A61K 31/375; A61K 31/122; A61K 31/51; A61K 31/525; A61K 31/455; A61K 31/4415; A61K 31/519; A61K 31/714; A61K 31/197; A61K 31/4188; A61K 35/741

(52) U.S. Cl. CPC A61K 36/45; A61K 35/741; A61K 47/44; A61K 31/702; A61K 33/00; A61K 33/06; A61K 33/26; A61K 33/34; A61K 33/32; A61K 33/18; A61K 33/24; A61K 33/04; A61K 31/07; A61K 31/593; A61K 31/355; A61K 31/375; A61K 31/122; A61K 31/51; A61K 31/525; A61K 31/455; A61K 31/4415; A61K 31/519; A61K 31/714; A61K 31/197; A61K 31/4188; A61K 38/1709

(57) ABSTRACT

A mixture (M) comprising or, alternatively consisting of: (i) water-soluble oligosaccharide, or a water-soluble complex carbohydrate, or mixtures thereof; (II) a casein; (ill) n3 and/or n6 fatty acids; (iv) an extract of Vaccinium macrocarpon. The present invention further relates to a pharmaceutical composition, or a food for special medical purpose (FSMP), or a medical device formulation or composition, or a dietary supplement comprising the aforementioned mixture (M), for use as medicament, or for use in the treatment of a chronic inflammatory bowel disease, in particular of states of malnutrition in subjects suffering from said chronic inflammatory bowel disease.

The present invention relates to a mixture comprising or, alternatively, consisting of a water-soluble saccharide, a casein, n3 and/or n6 fatty acids, and an extract of Vaccinium macrocarpon fruits.

Furthermore, the present invention relates to a pharmaceutical composition, or a food for special medical purpose (FSMP), or a medical device formulation or composition, or a dietary supplement (in short, the composition/s of the present invention) comprising the aforementioned mixture.

Furthermore, the present invention relates to the aforementioned composition for use in the treatment of a chronic inflammatory bowel disease selected from among the group comprising or, alternatively, consisting of Crohn's disease, ulcerative colitis, microscopic colitis (collagenosic colitis and lymphocytic colitis), eosinophilic esophagitis, indeterminate colitis, ischemic colitis, diversion colitis, Behçet's disease, pouchitis, ileitis and colitis.

Furthermore, the present invention relates to the aforementioned composition for use in the prevention or treatment of states of malnutrition in a subject suffering from a chronic inflammatory bowel disease. Lastly, the present invention relates to a method for preparing the aforementioned mixture.

Chronic inflammatory bowel diseases (IBDs) are a group of diseases that are characterised by inflammation of the gastrointestinal tract. Crohn's disease and ulcerative colitis are a type of IBD.

Subjects suffering from IBD may be at risk of malnutrition due to inadequate food intake, energy/protein imbalance, increased metabolic needs, and/or malabsorption.

Inadequate food intake may be caused by nausea, abdominal pain, loss of appetite or alteration of gustatory sensation.

Energy/protein imbalance be caused by protein loss, which is greater during exacerbations of intestinal inflammation, or fluid loss due to vomiting, fistula, diarrhoea or bleeding.

The metabolic needs can be increased by gastric inflammation, or by an infection superimposed on such inflammation, such as for example a fungal and/or bacterial infection (for example from enteroadherent Escherichia coli strains).

A malabsorption condition may be caused by the IBDs themselves, which affect more or less extensive sections of the small intestine. Malabsorption can also be aggravated by indispensable pharmacological treatments, or by bowel resection surgeries. Said malabsorption condition may in turn cause malnutrition in patients suffering from IBD, particularly in subjects with Crohn's disease and/or ulcerative or microscopic colitis.

In addition to an altered energy/protein balance, people suffering from IBD may be more at risk of deficiencies in a range of nutrients (such as vitamins, micronutrients and oligoelements), in shortage of which other essential physiological functions are also compromised.

As a result, proper management of the state of malnutrition in patients with IBD—each with different energy and nutritional needs—would facilitate a regular course of the disease, improve the absorption of nutritional components in subjects suffering from IBD, and prevent possible septic and/or post-operative complications.

Patients with IBD and suffering from nutritional deficiency may experience worsening disease prognosis, increased mortality and reduced quality of life.

Multicomponent compositions are known in literature, such as for example extracts of cranberry fruits, milk proteins, long chain and prebiotic fatty acids, indicated as compositions for maintaining or for restoring intestinal health (CN 109 645 500 A), dietary supplements or nutritional compositions (WO 2010/096564 A2, WO 2013/055439 A1) or used as “clinical nutrition” in patients suffering for example from HIV, cancer or diabetes (US 2006/269535 A1), or as compositions for the treatment of IBD (EP 2135616A1). At the same time, compositions for preventing and/or treating malnutrition which arise in subjects suffering from IBD due, for example, to a poor absorption of nutritional substances by these subjects, even in balanced and controlled diet conditions, are not known in the art.

The present invention therefore falls within the previous context, proposing a mixture or composition to be administered to IBD patients, such mixture or composition being developed in order to provide several specific and selected nutrients to IBD patients who may be malnourished or who, in the course of the disease, may incur a risk of malnutrition, in particular due to malabsorption of nutritional components which occurs in subjects with IBD, in particular in patients with Crohn's disease and/or ulcerative or microscopic colitis.

Thus, forming an object of the present invention is a mixture having the characteristics as defined in the attached claims.

Also forming an object of the present invention is a pharmaceutical composition, or a food for special medical purpose (FSMP), or a medical device formulation or composition, or a dietary supplement (in short, the composition/s of the present invention) comprising the aforementioned mixture, having the characteristics as defined in the attached claims.

Furthermore, forming an object of the present invention is a mixture or composition for use as medicament, in particular for use in a method for the treatment of a chronic inflammatory bowel disease and associated symptoms, wherein said chronic inflammatory bowel disease is selected from among the group comprising or, alternatively, consisting of Crohn's disease, ulcerative colitis, microscopic colitis (collagenosic colitis and lymphocytic colitis), eosinophilic esophagitis, indeterminate colitis, ischemic colitis, diversion colitis, Behçet's disease, pouchitis, ileitis and colitis, having the characteristics as defined in the attached claims.

Furthermore, forming an object of the present invention is the aforementioned composition for use in preventing and/or treating states of malnutrition in a subject suffering from a chronic inflammatory bowel disease (for example as defined above).

Lastly, forming an object of the present invention is a method for preparing the aforementioned mixture, having the characteristics as defined in the attached claims.

Preferred embodiments of the present invention will now be described below.

The present invention relates to a mixture (M) comprising or, alternatively, consisting of:

(i) a water-soluble oligosaccharide, or a water-soluble complex carbohydrate, or mixtures thereof (defined, together or separately, “water-soluble saccharide”);

(ii) a casein;

(iii) n3 and/or n6 fatty acids (or linseed oil);

(iv) an extract of Vaccinium macrocarpon fruits.

In other words, the component (i) of the present invention is a water-soluble saccharide selected from among the group comprising or, alternatively, consisting of: a water-soluble oligosaccharide, a water-soluble complex carbohydrate, and a mixture thereof.

According to an aspect of the invention, the water-soluble oligosaccharide (i), comprised in the mixture (M) of the invention together with (ii), (iii), (iv) and, optionally (i) a carbohydrate (according to any of the described embodiments), is at least one maltodextrin; said one or more maltodextrins preferably being a maltodextrin having CAS No. 9050-36-6.

Maltodextrins consisting of glucose molecules (or dextrose, an aldehyde monosaccharide) ordered in more or less long polymer chains are created based on the degree of transformation of starches. The length of the chains gives the parameter that allows to identify and classify the maltodextrins according to their dextrose equivalence (DE) starting from 2-4 to 20. The higher the DE, the shorter the polysaccharide chain. Therefore, maltodextrin will have a digestive behaviour more similar to that of glucose.

Sweetness is an important characteristic in the palatability of a food for special medical purposes and/or dietary supplement and, for example, taken as 100 the degree of sweetness of glucose, a maltodextrin with DE=17−19 has sweetness=14 whereas a maltodextrin with DE=4−10 has sweetness=11; another characteristic to be considered is the molecular weight since it affects the osmolarity of the solutions and therefore the gastric emptying time, hence a solution with 5.4% s.s. of d-glucose is isosmotic (300 mOsml/l), whereas a maltodextrin with DE 19 is isosmotic with 26.8% s.s.

Maltodextrins are easily digestible and they are also rapidly absorbed metabolically in the form of glucose. Maltodextrins are obtained by means of hydrolysis processes, mainly from the breakdown of starches from cereals (corn, oats, wheat, or rice) or tubers (potatoes, or tapioca).

Advantageously, the maltodextrins used in the present mixture (M) have an osmolarity comprised in a determined range, as defined below.

Osmolarity expresses the concentration of a solution, underlining the number of particles dissolved therein regardless of the electric charge and size. Osmolarity is expressed in osmoles per litre (osmol/l or OsM) or—when the solution is particularly diluted—in milliosmoles (mOsm) per litre (mOsm/l). For example, one litre of solution containing one mole of glucose will therefore have the same molarity as one litre of solution containing one mole of sodium (given that one mole, by definition, contains a fixed number of particles—atoms, ions or molecules—equal to 6.023×10\hat{ }23). However, the osmolarity of the two will be different from one litre of a third solution, containing one mole of kitchen salt; the latter (whose molecular formula is NaCl), in an aqueous medium, in fact dissociates itself into Na⁺ and Cl⁻, thus giving rise to a solution containing twice the particles. In the case of plasma osmolarity, under normal conditions, it is identical for all fluids present in the various compartments of the organism and its value is around 300 mOsm/l (possible gradients are nullified by water movements).

The maltodextrins used in the present mixture (M), preferably have osmolarity comprised in the range from 10 mOsm/l to 400 mOsm/l, preferably an osmolarity comprised from 50 mOsm/l to 350 mOsm/l, more preferably from 180 mOsm/l to 274 mOsm/l, even more preferably from 245 mOsm/l to 270 mOsm/l.

In the presence of a low osmolarity, maltodextrins are capable of passing more quickly through a gastric tract of a subject (or patient), and they are absorbed more rapidly in a subsequent intestinal tract due to favourable conditions of osmotic pressure present in the intestinal tract. This accelerated passage in the gastric tract results in a marked improvement of the digestive process. Improved digestion is certainly desirable in subjects suffering from chronic malabsorption and/or IBD.

Moreover, when ingested, maltodextrins do not pose particular problems of water flow into the intestinal lumen, nor of excessive increases in the subject's glycaemic index.

When dissolved in solution, the maltodextrins are also capable of providing a better suspendability of the mixture (M) (for example for probe applications), and give a better palatability to the mixture (M).

As a matter of fact, a problem frequently encountered in formulations of the prior art (especially in relation to formulations for elementary diets based on single amino acids) corresponding to the mixture (M) illustrated herein, relates precisely to palatability: if palatability is not optimal, a significant reduction in compliance with the nutritional plan may be envisaged. In other words, a non-optimal palatability leads to a greater probability that the subject fails to comply with the prescribed composition intakes, especially in cases where such compositions are used in an exclusive diet (as food).

The choice of all the components of the mixture (M) subject of the present invention, therefore not only of the water-soluble saccharide, therefore provided a painstaking analysis of palatability which is appreciated also by paediatric subjects.

A maltodextrin usable in the present mixture (M) is produced from corn starch, in the form of a white powder with a poured bulk density comprised from 380 g/l to 420 g/l, preferably ranging from 390 g/l to 410 g/l, and with a DE comprised from 18 to 20.

By way of example, maltodextrins usable in the present mixture (M), preferably produced by means of enzymatic hydrolysis, have the following amounts by weight of glucose, disaccharides and “higher” polysaccharides (i.e. higher than disaccharides), wherein said amounts are expressed as percentages by weight of the single component with respect to the total weight of the maltodextrins:
glucose: comprised from 0.1% to 5%, preferably comprised from 0.5% to 2%, even more preferably comprised from 0.8% to 1.2%, for example 0.8%, 0.9%, 1.0%, 1.1% or 1.2%;
disaccharides: comprised from 1% to 15%, preferably comprised from 2% to 10%, even more preferably comprised from 3% to 7%, for example 3%, 4%, 5%, 6% or 7%; and
higher polysaccharides: comprised from 85% to 98.9%, preferably comprised from 90% to 98%, even more preferably comprised from 92% to 96%), for example 92%, 94%, or 96%.

Preferably, the maltodextrins of the present mixture (M) have a loss on drying comprised from 0.1% to 10% (preferably comprised from 1% to 8%, even more preferably comprised from 3% to 7%), a protein content comprised from 0.01% to 1% (preferably comprised from 0.05% to 0.75%, even more preferably comprised from 0.1% to 3%), and a pH value in solution comprised from 4.0 to 6.0 (preferably comprised from 4.5 to 5.5).

For example, maltodextrins usable in the present mixture (M) are known under the trade name GLUCIDEX® it 19.

Alternatively or in addition to maltodextrins (one or more maltodextrins), the mixture (M) may comprise at least one water-soluble complex carbohydrate, which is selected from among the group comprising or, alternatively, consisting of an inulin, a starch and mixtures thereof.

Therefore, according to an embodiment of the invention, the mixture (M) comprises or, alternatively, consists of:

(i) a water-soluble saccharide selected from among:

(iaa) maltodextrin, or (ia) maltodextrin and inulin, or (ib) maltodextrin and starch, or (ic) maltodextrin, inulin and starch, or (id) inulin and starch; and

(ii) a casein; and

(iii) n3 and/or n6 fatty acids (or linseed oil); and

(iv) an extract of Vaccinium macrocarpon fruits.

Inulin is a natural polysaccharide synthesised by many plants and accumulated in the roots or rhizomes thereof. In industrial productions, inulin is mainly extracted from the common chicory (Cichorium intybus), from the tubers of Jerusalem artichokes and from black salsify (a kind of plant belonging to the family Asteraceae).

In the mixture (M), inulin is preferably an inulin having CAS No. 9005-80-5.

In an embodiment, the water-soluble saccharide (I), comprised in the mixture (M) of the invention together with (ii), (iii) and (iv) (according to any of the described embodiments), is a mixture of a maltodextrin having CAS No. 9050-36-6 and an inulin having CAS No. 9005-80-5, preferably at a weight ratio comprised from 1:5 to 5:1, preferably from 1:2 to 2:1, even more preferably 1:1.

Starch is a plant reserve carbohydrate, enzymatically synthesised from the glucose produced by chlorophyll photosynthesis. Starch consists of polymer units of amylose (which account for about 20% of the total polymer units) and amylopectin (which account for about 80% of the total polymer units).

In the mixture (M), starch is preferably a starch having CAS No. 9005-25-8.

In an embodiment, the water-soluble saccharide (i), comprised in the mixture (M) of the invention together with (ii), (iii) and (iv) (according to any of the described embodiments), is a mixture of a maltodextrin having CAS No. 9050-36-6, an inulin having CAS No. 9005-80-5, and a starch having CAS No. 9005-25-8, preferably at a 1:1:1 or 2:1:1, or 3:1:1 weight ratio.

Casein is a family of phosphoproteins found mainly in fresh milk, and which is the first source of milk proteins by abundance (about three-quarters of milk proteins are caseins).

Caseins include proteins and fats.

According to an aspect of the invention, the casein (ii) present in the mixture (M), together with (i), (iii) and (iv) (according to any of the described embodiments), contains proteins at an amount by weight comprised from 80% to 90% (N×6.38 on dry weight basis), preferably comprised from 83% to 87% by weight with respect to the total weight of casein, and fats at an amount by weight comprised from 0.1% to 10%, preferably comprised from 0.5% to 3%, with respect to the total weight of casein; preferably said casein has an ash residue at 550° C. comprised from 4% to 10% by weight, preferably from 5% to 8% by weight, with respect to the total weight of the casein; preferably said casein has a pH ranging from 6 to 8, preferably from 6.5 to 7.5; preferably said casein has a moisture content comprised from 0.5% to 2% by weight, preferably from 0.8% to 1.3% by weight, with respect to the total weight of casein.

Casein is considered an excellent protein source by virtue of the amino acid set that it consists of.

The present inventors have found that casein is capable of depressing pro-inflammatory patterns typical of IBD (and, more specifically, Crohn's disease, ulcerative colitis, microscopic colitis (collagenosic colitis and lymphocytic colitis), eosinophilic esophagitis, indeterminate colitis, ischemic colitis, diversion colitis, Behçet's disease, pouchitis, ileitis and colitis) when administered alone, but especially when administered in combination with the water-soluble saccharide (i) (as defined in the context of the present invention). According to an aspect of the invention, the casein (ii) contained in the mixture (M), together with (i), (iii) and (iv) (according to any of the described embodiments), is a micellar casein, i.e. in the native form thereof.

The casein (ii) usable in the present mixture (M), together with (i), (iii) and (iv) (according to any of the described embodiments), comprises casein and whey, preferably a [casein:whey] by weight ratio comprised from 85:15 to 99:1, preferably between 90:10 and 98:2, more preferably from 94:6 to 96:4, for example substantially at a ratio of 95:5.

The casein (ii) usable in the present mixture (M), together with (i), (iii) and (iv) (according to any of the described embodiments), may comprise or, alternatively, consist of milk proteins, whey proteins, whey protein isolate (in short, WPI).

For example, a casein (ii) present in the mixture (M), together with (i), (iii) and (iv) (according to any of the described embodiments), typically comprises the following amino acid composition, wherein the amounts of each amino acid are expressed in g of amino acid per 100 g of proteins: alanine 2.5-3.5 (preferably 3.1), arginine 3.0-4.0 (preferably 3.7), asparagine and/or aspartic acid 7.0-8.0 (preferably 7.4), cysteine 0.1-1.0 (preferably 0.5), glutamine and/or glutamic acid 21.0-24.0 (preferably 22.0-23.0, even more preferably 22.4), glycine 1.0-2.0 (preferably 1.8), histidine 2.0-3.0 (preferably 2.7), isoleucine 4.5-5.5 (preferably 5.2), leucine 8.0-11.0 (preferably 9.0-10.0, more preferably 9.5), lysine 7.5-8.5 (preferably 8.1), methionine 2.5-3.5 (preferably 2.9), phenylalanine 4.5-5.5 (preferably 5.2), proline 8.5-12.5 (preferably 9.5-11.5, even more preferably 10.4), serine 5.5-6.5 (preferably 5.8), threonine 4.0-5.0 (preferably 4.5), tryptophan 0.5-1.5 (preferably 1.2), tyrosine 5.0-6.0 (preferably 5.7), valine 6.0-7.0 (preferably 6.6).

More preferably, a casein (ii) usable in the present mixture (M) comprises the following mineral elements or salts, wherein the amounts of each mineral element or salt are expressed in g or mg of element per 100 g proteins: calcium comprised from 1 g to 4 g (preferably comprised from 1.8 g to 2.3 g), phosphorus comprised from 0.5 g to 3 g (preferably comprised from 1.0 g to 1.8 g), sodium comprised from 10 mg to 100 mg (preferably comprised from 30 mg to 50 mg), potassium comprised from 30 mg to 500 mg (preferably comprised from 120 mg to 200 mg), magnesium from 20 mg to 300 mg (preferably comprised from 50 mg to 150 mg), and chlorine from 10 mg to 150 mg (preferably comprised from 45 mg to 95 mg). In the mixture (M) of the invention (according to any of the described embodiments) the [water-soluble saccharide (i)]: [casein(ii)] by weight ratio is preferably comprised from 4:1 to 1:4, more preferably from 3:1 to 1:3, even more preferably from 2:1 to 1:2, for example 1:1.

In an embodiment of the mixture (M) comprising (i), (ii), (iii) and (iv) according to any of the described embodiments, (i) is a maltodextrin having CAS No. 9050-36-6 and (ii) is a micellar casein, preferably comprising casein and whey, more preferably at a casein: whey by weight ratio comprised from 85:15 to 99:1, even more preferably from 90:10 to 98:2, further preferably from 94:6 to 96:4, for example substantially at a ratio of 95:5.

In another embodiment of the mixture (M) comprising (i), (ii), (iii) and (iv) according to any of the described embodiments, (i) is a mixture of a maltodextrin having CAS No. 9050-36-6 and an inulin having CAS N° 9005-80-5, preferably having a weight ratio comprised from 1:5 to 5:1, preferably from 1:2 to 2:1, even more preferably 1:1, and (ii) is a micellar casein, preferably comprising casein and whey, more preferably at a casein: whey by weight ratio comprised from 85:15 to 99:1, even more preferably from 90:10 to 98:2, further preferably from 94:6 to 96:4, for example substantially at a ratio of 95:5.

In another embodiment of the mixture (M) comprising (i), (ii), (iii) and (iv) according to any of the described embodiments, (i) is a mixture of a maltodextrin having CAS No. 9050-36-6, an inulin having CAS No. 9005-80-5 and a starch having CAS No. 9005-25-8, preferably having a weight ratio of 1:1:1 or 2:1:1 or 3:1:1, and (ii) is a micellar casein, preferably comprising casein and whey, more preferably a casein: serum by weight ratio comprised from 85:15 to 99:1, even more preferably from 90:10 to 98:2, further preferably from 94:6 to 96:4, for example substantially at a ratio of 95:5.

Besides (i), (ii) and (iv), the aforementioned mixture (M) also comprises a linseed oil as a source of n3 and n6 fatty acids (iii).

Linseed oil comprises α-linolenic acid (n3 fatty acid) and linoleic acid (n6 fatty acid).

Linseed oil (or flax oil) is a lipid source, obtained from the seeds of Linum usitatissimum L. (of the Linaceae botanical family), highly rich in essential polyunsaturated fatty acids with anti-inflammatory action. By way of example, essential polyunsaturated fatty acids include α-linolenic acid (n3 fatty acid) and linoleic acid (n6 fatty acid), while semi-essential fatty acids include eicosapentaenoic acid 20:5 (EPA) (n3 fatty acid), docosahexaenoic acid (DHA) 22:6 (n3 fatty acid), linoleic acid 18:3 (GLA) (n6 fatty acid), dihomo-γ-linolenic acid (DGLA) 20:3 (n6 fatty acid), arachidonic acid 20:4 (AA) (n6 fatty acid), adrenic acid 22:4, docosapentaenoic acid 22:5 (n6 fatty acid).

According to one aspect of the invention, the α-linolenic acid: linoleic acid by weight ratio in linseed oil is comprised from 5:1 to 1:5, preferably from 4:1 to 1:4.

The percentage by weight of α-linolenic acid in linseed oil used in the present mixture (M) (comprising (i), (ii), (iii) and (iv) according to any of the described embodiments) as source of n3 and n6 fatty acids (iii) could be com 20% to 80% by weight, preferably comprised from 30% to 70% by weight, more preferably from 40% to 60% by weight.

According to an aspect of the invention, additionally or alternatively to the % by weight of α-linolenic acid in the linseed oil defined above, the percentage by weight of linoleic acid in the linseed oil of the present mixture (M) (comprising (i), (ii), (iii) and (iv) according to any of the described embodiments) is comprised from 5% to 25% by weight, preferably comprised from 10% to 20% by weight, more preferably from 12% to 19% by weight.

Further, besides linoleic acid and α-linolenic acid, components of the linseed oil used in the present mixture (M) could comprise palmitic acid (saturated fatty acid) at an amount comprised from 2% to 15% by weight, preferably comprised from 4% to 10%, stearic acid (saturated fatty acid) at an amount comprised from 1% to 10%, preferably comprised from 2.5% to 5.0%, and oleic acid (monounsaturated fatty acid) at an amount comprised from 5% to 40%, preferably comprised from 16% to 30%.

The linseed oil, usable in the context of the present invention, is preferably in the form of powder. In particular, such oil could be absorbed or adsorbed or incorporated in a carrier in the form of a powder, preferably an inert carrier in the form of spherical microparticles, even more preferably silicon dioxide (CAS No. 14464-46-1).

The percentage by weight of linseed oil is preferably comprised from 60% to 90% by weight, preferably comprised from 65% to 85% by weight, even more preferably comprised from 71% to 79% by weight, for example of 75% by weight, with respect to the total weight of such oil and the carrier in the form of powder. Preferably, the linseed oil in the form of powder could have a water activity (aW) comprised from 0.01 to 1.5, preferably comprised from 0.5 to 1.0, a pH value comprised from 3.5 to 8.5, preferably comprised from 4.5 to 7.5, and it could contain heavy metals at a total amount comprised from 0.1 ppm to 5 ppm (for example lead from 0.1 ppm to 3 ppm, cadmium from 0.1 ppm to 1 ppm, mercury from 0.01 ppm to 0.1 ppm).

More preferably, the linseed oil used in the present mixture (M) has an acidity of less than 1.0 mg KOH/g, and a density comprised from 0.927 g/cm3 to 0.937 g/cm3 measured at 20° C.

Even more preferably, the linseed oil used in the present mixture (M) has a peroxide number lower than 2.0 Meq O2/kg, an iodine number comprised from 166 g to 190 g 12/100 g, a saponification number comprised from 186 mg to 194 mg KOH/g, and an amount by weight of unsaponifiable matter comprised from 0.1% and 1.0% with respect to the total weight of said flax oil.

In the mixture (M) of the invention (according to any of the described embodiments) the [linseed oil (iii): casein(ii)] by weight ratio is preferably comprised from 3:1 to 1:3, more preferably from 1.5:1 to 1:1.5, even more preferably of about 1:1.

In the mixture (M) of the invention (according to any of the described embodiments) the [water-soluble saccharide (i)]: [casein(ii)] by weight ratio is preferably comprised from 4:1 to 1:4, more preferably from 3:1 to 1:3, even more preferably from 2:1 to 1:2, for example 1:1; and the [linseed oil (iii):casein(ii)] by weight ratio is preferably comprised from 3:1 to 1:3, more preferably from 1.5:1 to 1:1.5, even more preferably of about 1:1.

Vaccinium macrocarpon (also referred to by the synonyms of cranberry, small cranberry or swamp cranberry, or Vaccinium macrocarpon aiton, or Vaccinium macrocarpon L) is a small fruit plant of the Ericaceae family that produces red berries (the fruits).

In the aforementioned mixture (M), the extract of the Vaccinium macrocarpon fruits (iv) is used combined with (i), (ii) and (iii) (according to any of the described embodiments), wherein the source of n3 and/or n6 fatty acids is preferably linseed oil.

Preferably, the extract of the Vaccinium macrocarpon fruits (iv) used in the present mixture (M) is a titrated extract (Ph. EUR. 6.0; January 2008: 12 20) in proanthocyanidins. Preferably, the titrated extract of the Vaccinium macrocarpon fruits (iv), present in the mixture (M) together with (i), (ii) and (iii) (according to any of the described embodiments), is titrated in proanthocyanidins at a percentage by weight comprised from 10% to 60%, preferably from 30% to 50%, even more preferably from 35% to 45%, for example 36%, 38%, 40%, 42% or 44%.

For example, the amount of proanthocyanidins in 36 mg of the extract (iv) is preferably comprised from 5 mg to 100 mg, more preferably comprised from 8 mg to 70 mg, even more preferably comprised from 10 mg to 40 mg, further preferably comprised from 11 mg to 20 mg.

The extract (iv) could be a liquid extract or a dry extract, preferably it is an extract obtained from the Vaccinium macrocarpon fruit only.

The liquid extract (iv) is obtained through extraction using an extraction solvent comprising or, alternatively, consisting of water, or water and alcohol, preferably water and ethanol.

The dry extract (iv) is preferably obtained from the liquid extract by evaporating the extraction solvent, preferably under reduced pressure. As an alternative to evaporation at reduced pressure, the dry extract (iv) can be obtained by spray drying the liquid extract.

An extract (iv) usable in the present mixture (M), together with (i), (ii) and (iii) (according to any one of the described embodiments), is preferably in the form of a water-soluble powder (dry extract), with an average particle size distribution of powder comprised from 1 μm to 500 μm, preferably comprised from 10 μm to 400 μm, even more preferably comprised from 15 μm to 250 μm.

The dry extract (iv) preferably also contains one or more excipients at an amount comprised from 0.1% to 40% by weight, preferably comprised from 1% to 25%, with respect to the total weight of said extract. By way of example, the excipient could comprise or, alternatively, consist of maltodextrins, preferably maltodextrins obtained by breaking up corn starches.

More preferably, an extract (iv) usable in the present mixture (M), together with (i), (ii) and (iii) (according to any one of the described embodiments), has a pH value comprised from 1.0 to 5.5, preferably comprised from 2.0 to 4.5, a moisture content comprised from 0.1% to 5.0% by weight with respect to the total weight of said extract (iv), and a total heavy metal content comprised from 0.1 ppm to 5 ppm (for example lead from 0.1 ppm to 3 ppm, cadmium from 0.1 ppm to 1 ppm, mercury from 0.01 ppm to 0.1 ppm).

With regard to the extract (iv) of the present mixture (M), the present inventors obtained preliminary evidence indicating that the extract of Vaccinium macrocarpon fruits (iv) has an improved function in the treatment of chronic inflammatory bowel diseases, especially combined with the n3 and/or n6 fatty acids and with casein and/or a water-soluble saccharide. More precisely, in experimental models of induced colitis, such extract (iv) would have an efficacy in reducing the symptoms typical of colitis, a strong anti-inflammatory activity, a positive modulation of the intestinal microbiota, a positive modulation on the expression of cytokines with a proinflammatory phenotype and on the anti-inflammatory action. The anti-inflammatory action is carried out in particular through the modulation of the gene expression of proinflammatory mediators (such as TNF-α, IP-10, I-TAC, sICAM-1, GRO-α), also exerting a protective action in the induction of the active phases of the disease.

According to a preferred embodiment, the mixture (M) comprises:

(i) the water-soluble saccharide (preferably maltodextrins) at an amount comprised from 35% to 60% by weight, preferably from 40% to 58% by weight, more preferably from 45% to 55% by weight;

(ii) the casein, preferably micellar casein at an amount comprised from 10% to 30% by weight, preferably from 15% to 28% by weight, more preferably from 20% to 26% by weight;

(iii) the n3 and/or n6 fatty acids (preferably linseed oil as source of said acids) at an amount comprised from 3% to 20% by weight, preferably from 5% to 16% by weight, more preferably from 7% to 14% by weight;

(iv) the extract of Vaccinium macrocarpon fruits at an amount comprised from 0.01% to 5% by weight, preferably from 0.05% to 3% by weight, even more preferably from 0.1% to 1% by weight;

in which % by weight are expressed with respect to the total weight of the mixture (M)

Forming an object of the present invention is a pharmaceutical composition or a food for special medical purpose (FSMP), or a medical device formulation or composition, or a dietary supplement (in short, composition of the invention or composition (c)), comprising (a) said mixture (M) and (e) at least one physiologically and/or pharmacologically acceptable technological additive or excipient.

Further, forming an object of the present invention is a pharmaceutical composition or a food for special medical purpose (FSMP), or a medical device formulation or composition, or a dietary supplement, comprising (a) said mixture (M), (b) one or more mineral elements or salts, (d) one or more vitamin substances, (e) at least one physiologically and/or pharmacologically acceptable technological additive or excipient.

The mineral elements or salts (b), comprised in the composition of the invention together with the mixture (M) and (e) and, optionally, (d) (according to any of the described embodiments), are preferably selected from among the group comprising or, alternatively, consisting of: potassium, calcium, magnesium, iron, copper, zinc, manganese, iodine, molybdenum, selenium, chromium, chlorine, sodium and combinations thereof.

Therefore, according to an embodiment of the present invention, the composition (c) comprises:

(a) the mixture comprising or, alternatively consisting of: (i) the water-soluble saccharide (preferably maltodextrins) at an amount preferably comprised from 35% to 60% by weight, more preferably from 40% to 58% by weight, even more preferably from 45% to 55% by weight; (ii) casein, preferably micellar casein, at an amount preferably comprised from 10% to 30% by weight, more preferably from 15% to 28% by weight, even more preferably from 20% to 26% by weight; (iii) the n3 and/or n6 fatty acids (preferably linseed oil as source of said acids) at an amount preferably comprised from 3% to 20% by weight, more preferably from 5% to 16% by weight, even more preferably from 7% to 14% by weight; (iv) the extract of Vaccinium macrocarpon fruit (preferably a dry extract) at an amount preferably comprised from 0.01% to 5% by weight, more preferably from 0.05% to 3% by weight, even more preferably from 0.1% to 1% by weight;

(b) one or more mineral elements or salts selected from among the group comprising or, alternatively consisting of: potassium, calcium, magnesium, iron, copper, zinc, manganese, iodine, molybdenum, selenium, chromium, chlorine, sodium and the combinations thereof;

(d) one or more vitamin substances;

(e) at least one physiologically and/or pharmacologically acceptable technological additive or excipient.

The selection, quantity and quality of mineral salts were identified based on the need to develop a nutritionally complete composition (c); the mineral salts were developed based on the specific needs of patients with IBD, and deficits identified in studies evaluating the state of nutrition in patients with Crohn's disease, ulcerative colitis, microscopic colitis (collagenosic colitis and lymphocytic colitis), eosinophilic esophagitis, indeterminate colitis, ischemic colitis, diversion colitis, Behçet's disease, pouchitis, ileitis or colitis.

Such proportions make the composition (c) subject of the present invention extremely versatile in use, as a food, drug, medical device and supplement, especially due to the improved palatability, the activation of metabolic processes and the physiological functions toward which mineral salts contribute.

The vitamin substances (d), comprised in the composition of the invention together with the mixture (M) and (e) and, optionally, (c) (according to any of the described embodiments), are preferably selected from among the group comprising or, alternatively, consisting of: vitamin A (or retinol; CAS N. 68-26-8; preferably retinyl acetate), cholecalciferol (or vitamin D3), vitamin E (preferably DL-alpha-tocopheryl acetate), L-ascorbic acid (or vitamin C), vitamin K2 (preferably phytomenadione), vitamin B1 (or thiamine; preferably thiamine hydrochloride), vitamin B2 (or riboflavin), vitamin B3 (or nicotinamide CAS N. 98-92-0), vitamin B6 (preferably pyridoxine hydrochloride), folic acid or pteroylmonoglutamic acid (CAS No. 59-30-3), vitamin B12 (or cobalamin; preferably cyanocobalamin), vitamin H (CAS No. 58-85-5; preferably D-biotin), vitamin B5 (preferably calcium D-pantothenate), vitamin J (or choline; CAS No. 62-49-7, preferably choline bitartrate), vitamin B7 (or inositol; CAS No. 6917-35-7), and combinations thereof.

Therefore, according to an embodiment of the present invention, the composition (c) comprises:

(a) the mixture comprising or, alternatively consisting of: (i) the water-soluble saccharide (preferably maltodextrins) at an amount preferably comprised from 35% to 60% by weight, more preferably from 40% to 58% by weight, even more preferably from 45% to 55% by weight; (ii) casein, preferably micellar casein, at an amount preferably comprised from 10% to 30% by weight, more preferably from 15% to 28% by weight, even more preferably from 20% to 26% by weight; (iii) the n3 and/or n6 fatty acids (preferably linseed oil as source of said acids) at an amount preferably comprised from 3% to 20% by weight, more preferably from 5% to 16% by weight, even more preferably from 7% to 14% by weight; (iv) the extract of Vaccinium macrocarpon fruit (preferably a dry extract) at an amount preferably comprised from 0.01% to 5% by weight, more preferably from 0.05% to 3% by weight, even more preferably from 0.1% to 1% by weight;

(b) one or more mineral elements or salts, preferably selected from among the group comprising or, alternatively, consisting of: potassium, calcium, magnesium, iron, copper, zinc, manganese, iodine, molybdenum, selenium, chromium, chlorine, sodium and combinations thereof;

(d) one or more vitamin substances selected from among the group comprising or, alternatively, consisting of: vitamin A, retinyl acetate, vitamin D3, vitamin E, DL-alpha-tocopheryl acetate, vitamin C, vitamin K2, phytomenadione, vitamin B1, thiamine hydrochloride, vitamin B2, vitamin B3, vitamin B6, pyridoxine hydrochloride, folic acid, vitamin B12, cyanocobalamin, vitamin H, D-biotin, vitamin B5, calcium D-pantothenate, vitamin J, choline bitartrate, vitamin B7, and combinations thereof;

(e) at least one physiologically and/or pharmacologically acceptable technological additive or excipient.

The combination of the mixture (M) and vitamin substances (d) allows to supplement a range of nutrients necessary for a variety of metabolic functions, speeding up their absorption especially in people at risk of malnutrition.

A cholecalciferol usable in the present composition (c) is a powder with a loss on drying (measured using a gravimetry; at 105° C.) 5%. Such powder preferably has a heavy metal content 10 mg/kg.

A vitamin E usable in the present composition (c) is preferably a flowing powder, with a drying residue equal to or less than 5%.

An L-ascorbic acid usable in the present composition (c) is a crystalline powder preferably with a melting point of about 190° C. The pH value of a 5% aqueous solution of such powder is preferably comprised from 2.1 to 2.6.

Vitamin K2, usable in the present composition (c), preferably comprises a mixture of menaquinone-7 and menaquinone-6. Preferably, the menaquinone-7: menaquinone-6 by weight ratio in vitamin K2 is comprised from 120:1 to 20:1, more preferably from 100:1 to 30:1. Vitamin K2 is preferably micro-encapsulated. More preferably, menaquinone-7 and menaquinone-6 are mixed with a carrier of gum arabic (from 74% to 76% by weight) and sunflower oil (from 23% to 26% by weight).

A thiamine usable in the present composition (c) is preferably in the form of a water-soluble powder. Such powder preferably has a water content comprised from 0.1% to 5% by weight More preferably, an aqueous solution of such powder has a pH value comprised from 2.7 to 3.3.

A nicotinamide usable in the present composition (c) is preferably in a water-soluble powder, wherein the pH value of the aqueous solution of such powder is preferably comprised from 6.0 and 7.5. More preferably, a percentage by weight ≥90% of the particles of the nicotinamide powder has an average particle size ≥50 μm, and a percentage by weight 8% of the particles of the nicotinamide powder which has an average particle size ≥250 μm.

A pyridoxine hydrochloride usable in the present composition (c) is in the form of a white powder with a loss on drying ≤0.5%. Preferably, a 5% solution by weight of such powder has a pH value comprised from 2.4 to 3.0.

A riboflavin usable in the present composition (c) is preferably a crystalline fine powder with a loss on drying comprised from 0.1% to 1.5%, and more preferably with a residue on ignition comprised from 0.01% to 0.3%.

A vitamin H usable in the present composition (c) is preferably a powder soluble in water and alcohol, and more preferably with a loss on drying 10%.

A calcium D-pantothenate usable in the present composition (c) is preferably in the form of powder. A solution in water of such powder preferably has a pH value comprised from 6.8 to 8.0. More preferably, said powder has a loss on drying comprised from 0.1% to 3.0% and, even more preferably, it comprises chlorides at an amount equal to or less than 200 ppm.

An inositol usable in the present composition (c) is preferably a crystalline powder with a melting point comprised from 224.0° C. to 227.0° C., more preferably with a residue on ignition comprised from 0.01% a 0.1%.

A folic acid usable in the present composition (c) is preferably a crystalline powder, with a water content comprised from 0.1% to 15%, preferably comprised from 5% to 10%, and with a residue on ignition comprised from 0.1% to 0.3%.

A vitamin A usable in the present composition (c) is preferably a powder with a loss on drying 8.0%.

According to an embodiment, the composition (c) of the invention (according to any of the described embodiments) could be used in association with at least one isolated strain of bacteria, preferably bacteria with probiotic function, more preferably lactic ferments, and/or bacterial lysates, tyndallized bacteria, inactivated bacteria (paraprobiotic), postbiotics, or the combinations thereof. Therefore, according to such embodiment, one or more bacterial strains are used combined with the composition (c) comprising the mixture (M), the mineral salts (b), the vitamin substances (d), and the physiologically and/or pharmacologically acceptable technological additive or excipient (e), or such bacterial strains are present in the composition (c).

The weight ratio between the composition (c) (comprising the mixture (M) and (e) and, optionally, (b) and/or (d), or, alternatively, comprising the mixture (M), (b), (d) and (e)) and the at least one isolated bacterial strain is preferably comprised from 20:1 to 1:20, more preferably from 10:1 to 1:10, even more preferably from 5:1 to 1:5.

The present invention also relates to a method for preparing said mixture (M).

The method for preparing such mixture (M) comprises at least one step of mixing: (I) a water-soluble oligosaccharide, or a water-soluble complex carbohydrate, or a mixture thereof (water-soluble saccharide);

(ii) a casein; (iii) n3 and/or n6 fatty acids (or linseed oil); (iv) an extract of Vaccinium macrocarpon fruits. As regards the methods of use and administration of the mixture (M) and of the composition (c) subject of the present invention, no specific limitations are envisaged. However, a specific posology for each subject is referred to the advice of the treating physician, based on careful analysis of age, body weight, general health status, course of the subject's IBD disease, the type of IBD and the level of malnutrition the subject is suffering from.

The aforementioned composition (c) can be administered through the oral and/or through the enteral route.

The following Example 1 reports a composition (c) which can be administered through the oral route, according to an embodiment. Preferably, an amount of the composition (c) of example 1 can be dissolved in about 250 ml of water preferably uncarbonated, and then drunk.

In an administration through the enteral route, the composition (c) is preferably administered exclusively, i.e. in the absence of other enteral feeding compositions. In an administration through the enteral route, the amount of the composition (c) of Example 1 can be pre-dissolved in about 250 ml of uncarbonated water to obtain a solution of composition. A probe is then pre-washed with about 30 ml of water, the composition solution is administered through the probe, and lastly the probe is washed with other 30 ml of water.

Embodiments (FRn) according to invention are indicated below:

FR1. A mixture (M) comprising or, alternatively consisting of:

(i) a water-soluble oligosaccharide, or a water-soluble complex carbohydrate, or the mixtures thereof (water-soluble saccharide);

(ii) a casein;

(iii) n3 and/or n6 fatty acids;

(iv) an extract of Vaccinium macrocarpon fruits.

FR2. The mixture (M) according to the preceding FR, wherein the [water-soluble saccharide (i)]: [casein (ii)] by weight ratio is comprised from 4:1 to 1:4, preferably from 3:1 to 1:3, more preferably from 2:1 to 1:2.

FR3. The mixture (M) according to any one of the preceding FRs, comprising:
(i) the water-soluble saccharide at an amount comprised from 35% to 60% by weight, preferably from 40% to 58% by weight, more preferably from 45% to 55% by weight;

(ii) the casein, preferably micellar casein at an amount comprised from 10% to 30% by weight, preferably from 15% to 28% by weight, more preferably from 20% to 26% by weight;

(iii) the n3 and/or n6 fatty acids at an amount comprised from 3% to 20% by weight, preferably from 5% to 16% by weight, more preferably from 7% to 14% by weight;

(iv) the extract Vaccinium macrocarpon fruits at an amount comprised from 0.01% to 5% by weight, preferably from 0.05% to 3% by weight, more preferably from 0.1% to 1% by weight;

preferably the extract (iv) being titrated in proanthocyanidin at a percentage by weight comprised from 10% to 60%, preferably from 30% to 50%, even more preferably from 35% to 45%.

FR4. The mixture (M) according to any one of the preceding FRs, wherein the water-soluble oligosaccharide is a maltodextrin, and/or wherein the water-soluble complex carbohydrate is selected from among the group comprising or, alternatively consisting of inulin, starch and the mixtures thereof.

FR5. The mixture (M) according to one of the preceding FRs, comprising linseed oil as source of n3 and n6 fatty acids (iii), the linseed oil comprising α-linolenic acid and linolenic acid, the percentage by weight of the α-linolenic acid in the linseed oil being comprised from 20% to 80% by weight, preferably comprised from 30% to 70% by weight, more preferably from 40% to 60% by weight.

FR6. The mixture (M) according to the preceding FR, wherein the [linseed oil casein (ii)] by weight ratio is comprised from 3:1 to 1:3, preferably from 1.5:1 a 1:1.5, more preferably of about 1:1.

FR7. A pharmaceutical composition (c), or a food for special medical purpose (FSMP), or a medical device formulation or composition, or a dietary supplement, comprising the mixture (M) according to any one of the preceding claims, (b) one or more mineral salts, (d) one or more vitamin substances, (e) at least one physiologically and/or pharmacologically acceptable technological additive or excipient.

FR8. The composition (c) according to the preceding FR, wherein the mineral salt is selected from among the group comprising or, alternatively consisting of: potassium, calcium, magnesium, iron, copper, zinc, manganese, iodine, molybdenum, selenium, chromium, chlorine, sodium and the combinations thereof.

FR9. The composition (c) according to any one of FR7 or FR8, wherein the vitamin substance is selected from among the group comprising or, alternatively consisting of: vitamin A, vitamin D3, vitamin E, vitamin C, vitamin K2, vitamin B1, vitamin B2, vitamin B3, vitamin B6, folic acid, vitamin B12, vitamin H, vitamin B5, vitamin J, vitamin B7, and the combinations thereof.

FR10. The composition (c) according to any one of FR7-9, in association with at least one isolated strain of bacteria, preferably bacteria with probiotic function, more preferably lactic ferments, and/or bacterial lysates, tyndallised bacteria, postbiotics bacteria, or the combinations thereof.

FR11. The composition (c) according to any one of FR7-10, for use as medicament, or for use in the treatment of a chronic inflammatory bowel disease selected from among the group comprising or, alternatively consisting of Crohn's disease, ulcerative colitis, microscopic colitis, eosinophilic oesophagitis, indeterminate colitis.

FR12. Composition (c) according to any one of FR7-10 for use in the prevention or treatment of states of malnutrition in a subject suffering from a chronic inflammatory bowel disease.

FR13. A method for preparing a mixture (M) according to any one of FR1-6, comprising at least one step of mixing:

(i) a water-soluble oligosaccharide, or a water-soluble complex carbohydrate, or the mixtures thereof (water-soluble saccharide);

(ii) a casein;

(iii) n3 and/or n6 fatty acids;

(iv) an extract of Vaccinium macrocarpon fruits.

Unless specified otherwise, the expression composition or mixture or other comprising a component at an amount “comprised in a range from x to y” is used to indicate that said component can be present in the composition or other at all the amounts present in said range, even though not specified, extremes of the range comprised.

Unless specified otherwise, the indication that a composition “comprises” one or more components or substances means that other components or substances can be present besides the one, or the ones, indicated specifically.

In the context of the present invention, the expression “method for treatment” or “treatment” is used to indicate an intervention on a subject in need, comprising the administration of “a therapeutically effective amount” of a composition of the invention with the aim of eliminating, reducing/decreasing or preventing a disease or ailment and symptoms or disorders thereof.

In the context of the present invention, the term “subject/s” or “patients” is used to indicate mammals (animals and humans), preferably human, male and female subjects.

The term “therapeutically effective amount” refers to the amount of active compound and/or bacterial strain that elicits the biological or medicinal response in a tissue, system, mammal, or human being that is sought and defined by an individual, researcher, veterinarian, physician, or other clinician or health worker. In the context of the present invention, the term “medical device” is used at least in the meaning according to the Legislative Decree n° 46 dated 24 Feb. 1997, or in accordance with the new Medical device regulation (EU) 2017/745 (MDR).

In the context of the present invention, the term “novel food” is used at least in the meaning according to Regulation EC 258 dated 1997.

A non-limiting example of the present invention will be described below.

### EXAMPLES

### Example 1: Qualitative and Quantitative Composition of the Present Composition

Table 1 below illustrates an embodiment of the present composition (c).

TABLE 1

| Component | Weight in mg. |
|---|---|
| Maltodextrins (CAS N° 9050-36-6) | 13844.769 |
| micellar casein (sunflower lecithin) | 6875.000 |
| Linseed oil (Linum usitatissimum L., silicon dioxide) | 6666.667 |
| potassium citrate (CAS N° 866-84-2) | 430.556 |
| calcium phosphate (CAS No° 7758-87-4) | 368.966 |
| magnesium citrate (CAS N° 7779-25-1) | 262.500 |
| flavour (for example vanilla) | 250.00 |
| choline bitartrate (CAS N° 87-67-2) | 83.750 |
| sodium chloride (CAS N° 7647-14-5) | 82.562 |
| sweetener: sucralose (CAS N° 56038-13-2) | 45.00 |
| extract of Vaccinium macrocarpon L fruit titrated to | 36.250 |
| 40% proanthocyanidins | |
| L-ascorbic acid (CAS N° 50-81-7) | 18.500 |
| iron pyrophosphate (CAS N° 10058-44-3) | 11.765 |
| DL-alpha-tocopheryl acetate (CAS N° 7695-91-2) | 7.778 |
| zinc citrate (CAS N° 546-46-3) | 4.968 |
| phytomenadione (glucose syrup, gum arabic, | 2.500 |
| phytomenadione, tricalcium phosphate) | |
| Inositol (CAS N° 6917-35-7) | 2.400 |
| Nicotinamide (CAS N° 98-92-0) | 2.250 |
| manganese gluconate (CAS N° 6485-39-8) | 0.957 |
| retinyl acetate (CAS N° 127-47-9) | 0.517 |
| calcium d-pantothenate (CAS N° 137-08-6) | 0.488 |
| copper sulphate (CAS No° 7758-98-7) | 0.431 |
| Cholecalciferol (sucrose, gum arabic, corn starch, | 0.350 |
| medium chain triglycerides, tricalcium phosphate, | |
| cholecalciferol, DL-alpha-tocopherol) | |
| pyridoxine hydrochloride (CAS N° 58-56-0) | 0.207 |
| thiamine hydrochloride (CAS N° 67-03-8) | 0.148 |
| Riboflavin (CAS N° 83-88-5) | 0.115 |
| pteroylmonoglutamic acid (CAS N° 59-30-3) | 0.039 |
| Chromium picolinate (CAS N° 14639-25-9) | 0.038 |
| sodium selenite (CAS N° 10102-18-8) | 0.021 |
| potassium iodide (CAS N° 7681-11-0) | 0.012 |
| sodium molybdate (CAS N° 10102-40-6) | 0.010 |
| D-biotin (CAS N° 58-85-5) | 0.005 |
| Cyanocobalamin (CAS N° 68-19-9) | 0.000234 |
| Total weight: | 29000.00 |

A potassium citrate, preferably anhydrous, usable in the present composition (c) is preferably a powder with a loss on drying at (180° C. for four hours) comprised from 0.5% to 2.0%.

The calcium phosphate is preferably anhydrous and in powder form. More preferably, such powder has a loss on ignition at 800° C. comprised from 7.0% to 8.5%, and a loss on drying (at 150° C. for two hours) comprised from 1.0% to 3.0%. Even more preferably, a solution in water containing 20% by weight of such powder has a pH value comprised from 4.5 to 6.0.

A magnesium citrate, preferably dibasic, usable in the present composition (c) is a water-soluble fine powder, in which an aqueous solution at 5% by weight of such powder has a pH value comprised from 3.7 to 4.0. Preferably, such powder could contain heavy metals at a total amount comprised from 0.1 ppm to 5 ppm (for example lead from 0.1 ppm to 3 ppm, cadmium from 0.1 ppm to 1 ppm, mercury from 0.01 ppm to 0.1 ppm).

A sodium chloride usable in the present composition (c) is in the form of crystals, with a titre comprised from 95% to 100%, and with a density comprised from 1150 g/l to 1220 g/l.

A sucralose usable in the present composition (c) is a powder a calcination residue equal to or less than 0.7%. A 10% aqueous solution of such powder preferably has a pH value comprised from 5.0 to 7.0.

A zinc citrate usable in the present composition (c) is preferably in the form of powder, more preferably with an amount of zinc comprised from 30.0% to 32.5% by weight with respect to the total weight of the powder.

Preferably, a choline bitartrate usable in the present composition (c) is a crystalline powder which, when solubilized at a concentration of 10% by weight in water, has a pH value comprised from 3.0 to 4.0.

Preferably, an iron pyrophosphate usable in the present composition (c) is a powder comprising or, alternatively, consisting of ferric pyrophosphate, rapeseed lecithin (CAS No. 8002-43-5), sodium chloride and maltodextrins. Preferably, the iron content in such powder is comprised from 75 mg to 95 mg for each gram of powder, preferably comprised from 80 mg/g to 90 mg/g. Even more preferably, this powder has a density comprised from 0.6 g/ml to 1.0 g/ml, and a loss on drying comprised from 1% to 10%.

A manganese gluconate, preferably dihydrate, usable in the present composition (c) is a powder with a water content preferably comprised from 6.0% to 9.0% by weight, and more preferably with a heavy metal content (for example lead) at a total amount comprised from 0.0005% to 0.001% ppm.

A copper sulphate, preferably pentahydrate, usable in the present composition (c) has a copper titre (TG415 5.2.1 method) 25.30%. Preferably, a 5% aqueous solution by weight of such sulphate has a pH value comprised from 3.70 to 4.20.

A cyanocobalamin a usable in the present composition (c) is a powder preferably with a loss on drying comprised between 0.1% at 3.0%. More preferably, a residual content of solvents (for example acetone) in such powder is 5.000 ppm.

A chromium picolinate usable in the present composition (c) is preferably a powder insoluble in water and in alcohol, more preferably with a melting point comprised from 245° C. to 253° C. Such powder preferably has a bulk density comprised from 0.3 g/ml to 0.55 g/ml, preferably comprised from 0.4 g/ml to 0.5 g/ml.

A sodium selenite, preferably anhydrous, usable in the present composition (c) is preferably a crystalline powder with a loss on drying at (130° C. for two hours) comprised from 0.1% to 0.5%. Preferably, a 5% aqueous solution by weight of such powder has a pH value comprised from 9.8 to 10.8.

A potassium iodide usable in the present composition (c) is in the form of crystals preferably characterised by a loss on drying comprised from 0.1% to 1.0%. Preferably, such crystals have a heavy metal content comprised from 0.1 ppm to 10 ppm.

A sodium molybdate usable in the present composition (c) is preferably a crystalline solid which breaks up at temperatures higher than 100° C. with formation of anhydride. Preferably, said crystalline solid has a molybdenum content comprised from 35% to 45% by weight, preferably comprised from 39.5% to 40.5% by weight.

The embodiments of the mixture, of the composition comprising such mixture, of said composition for use as medicament or for use in the treatment of a chronic inflammatory bowel disease or a state of malnutrition in subjects suffering from a chronic inflammatory bowel disease (e.g. Crohn's disease, ulcerative or microscopic colitis), and of the aforementioned method shall be subjected—by a man skilled in the art—to substitutions or changes as relates to the described characteristics depending on the contingency. These embodiments are also to be considered included in the scope of protection formalised in the following claims.

Furthermore, it should be observed that any embodiment may be implemented independently from the other embodiments described.

1. A mixture (M) for use in a method for preventive and/or curative treatment of a state of malnutrition in a subject suffering from a chronic inflammatory bowel disease,
wherein said mixture (M) comprises or, alternatively, consists of:
(i) a water-soluble saccharide selected from among a water-soluble oligosaccharide, a water-soluble complex carbohydrate, or mixtures thereof;
(ii) a casein;
(iii) a linseed oil as source of n3 and/or n6 fatty acids, wherein said linseed oil comprises a α-linolenic acid and a linoleic acid, wherein the α-linolenic acid is comprised in the linseed oil at a % by weight comprised in the range from 20% to 80%, with respect to the total weight of linseed oil; and
(iv) an extract of Vaccinium macrocarpon fruits;
and wherein the mixture (M) comprises:
(i) the water-soluble saccharide at an amount comprised in the range from 35% to 60% by weight;
(ii) the casein at an amount comprised in the range from 10% to 30% by weight;
(iii) the n3 and/or n6 fatty acids at an amount comprised in the range from 3% to 20% by weight;
(iv) the extract of Vaccinium macrocarpon fruits at an amount comprised in the range from 0.01% to 5% by weight, the extract (iv) being titrated in proanthocyanidins at a % by weight comprised from 10% to 60% by weight; wherein said % are expressed with respect to the total weight of the mixture (M).

2. The mixture (M) for use according to claim 1, wherein said chronic inflammatory bowel disease is selected from among: Crohn's disease, ulcerative colitis, microscopic colitis, collagenous colitis and lymphocytic colitis, eosinophilic esophagitis, and indeterminate colitis, ischemic colitis, diversion colitis, Behçet's disease, pouchitis, ileitis and colitis.

3. The mixture (M) according to claim 1, wherein the water soluble saccharide (i): casein (ii) by weight ratio is comprised from 4:1 to 1:4, preferably from 3:1 to 1:3, more preferably from 2:1 to 1:2.

4. The mixture (M) according to claim 1, comprising:
(i) the water-soluble saccharide at an amount comprised in the range from 40% to 58% by weight, preferably from 45% to 55% by weight;
(ii) the casein, preferably micellar casein, at an amount comprised in the range from 15% to 28% by weight, preferably from 20% to 26% by weight;
(iii) the n3 and/or n6 fatty acids at an amount comprised in the range from 5% to 16% by weight, preferably from 7% to 14% by weight;
(iv) the extract of Vaccinium macrocarpon fruits at an amount comprised in the range from 0.05% to 3% by weight, preferably from 0.1% to 1% by weight; the extract (iv) being titrated in proanthocyanidins at a percentage by weight comprised in the range from 30% to 50% by weight, preferably from 35% to 45% by weight; wherein said % are expressed with respect to the total weight of the mixture (M).

5. The mixture (M) according to claim 1, wherein the water-soluble oligosaccharide is a maltodextrin, and/or wherein the water-soluble complex carbohydrate is selected from among the group comprising or, alternatively, consisting of inulin, starch and mixtures thereof.

6. The mixture (M) according to claim 1, wherein the percentage by weight of α-linolenic acid in the linseed oil is comprised in the range from 30% to 70% by weight, preferably from 40% to 60% by weight, with respect to the total weight of the linseed oil.

7. The mixture (M) according to the claim 6, wherein the linseed oil:
casein (ii) by weight ratio is comprised from 3:1 to 1:3, preferably from 1.5:1 and 1:1.5, more preferably of about 1:1.

8. A method for preventive and/or curative treatment of a state of malnutrition in a subject suffering from a chronic inflammatory bowel disease, comprising administering to the subject a composition comprising mixture (M) comprising:
(i) a water-soluble saccharide selected from among a water-soluble oligosaccharide, a water-soluble complex carbohydrate, or mixtures thereof;
(ii) a casein;
(iii) a linseed oil as source of n3 and/or n6 fatty acids, wherein said linseed oil comprises a α-linolenic acid and a linoleic acid, wherein the α-linolenic acid is comprised in the linseed oil at a % by weight comprised in the range from 20% to 80%, with respect to the total weight of linseed oil; and
(iv) an extract of Vaccinium macrocarpon fruits; wherein
(i) the water-soluble saccharide is at an amount comprised in the range from 35% to 60% by weight;
(ii) the casein is at an amount comprised in the range from 10% to 30% by weight;
(iii) the n3 and/or n6 fatty acids is at an amount comprised in the range from 3% to 20% by weight;
(iv) the extract of Vaccinium macrocarpon fruits is at an amount comprised in the range from 0.01% to 5% by weight, the extract (iv) being titrated in proanthocyanidins at a % by weight comprised from 10% to 60% by weight; wherein said % are expressed with respect to the total weight of the mixture (M).

9. The method of claim 8, wherein said patient is suffering from a chronic inflammatory bowel disease selected from among Crohn's disease, ulcerative colitis, microscopic colitis, eosinophilic esophagitis, and indeterminate colitis.

10. The method of claim 8, wherein said composition further comprises (b) one or more mineral salts, wherein the mineral salt is selected from among the group comprising or, alternatively, consisting of: potassium, calcium, magnesium, iron, copper, zinc, manganese, iodine, molybdenum, selenium, chromium, chlorine, sodium and combinations thereof.

11. The method of claim 8, wherein said composition further comprises (d) one or more vitamin substances, wherein the vitamin substance is selected from among the group comprising or, alternatively, consisting of: vitamin A, vitamin D3, vitamin E, vitamin C, vitamin K2, vitamin B1, vitamin B2, vitamin B3, vitamin B6, folic acid, vitamin B12, vitamin H, vitamin B5, vitamin J, vitamin B7, and combinations thereof.

12. The method of claim 8, in association with at least one isolated strain of bacteria, preferably bacteria with probiotic function, more preferably lactic ferments, and/or bacterial lysates, tyndallized bacteria, inactivated bacteria (paraprobiotics), postbiotics, or combinations thereof.

13. The method of claim 8, wherein said composition is a pharmaceutical composition, or a food for special medical purpose (FSMP), or a medical device formulation or composition, or a dietary supplement.

14. The method of claim 8, wherein the water soluble saccharide (i): casein (ii) by weight ratio is in the range of 4:1 to 1:4.

15. The method of claim 8, wherein
(i) the water-soluble saccharide is at an amount comprised in the range from 40% to 58% by weight, preferably from 45% to 55% by weight;
(ii) the casein, preferably micellar casein, is at an amount comprised in the range from 15% to 28% by weight, preferably from 20% to 26% by weight;
(iii) the n3 and/or n6 fatty acids are at an amount comprised in the range from 5% to 16% by weight, preferably from 7% to 14% by weight;
(iv) the extract of Vaccinium macrocarpon fruits is at an amount comprised in the range from 0.05% to 3% by weight, preferably from 0.1% to 1% by weight; the extract (iv) being titrated in proanthocyanidins at a percentage by weight comprised in the range from 30% to 50% by weight, preferably from 35% to 45% by weight; wherein said % are expressed with respect to the total weight of the mixture (M).

16. The method of claim 8, wherein the water-soluble oligosaccharide is a maltodextrin, and/or wherein the water-soluble complex carbohydrate is selected from among the group comprising or, alternatively, consisting of inulin, starch and mixtures thereof.

17. The method of claim 8, wherein the percentage by weight of α-linolenic acid in the linseed oil is comprised in the range from 30% to 70% by weight, with respect to the total weight of the linseed oil.

18. The method of claim 8, wherein the linseed oil: casein (ii) by weight ratio is comprised from 3:1 to 1:3.
